(19) United States
(12) Patent Application Publication
(10) Pub. No.: US 2010/0318098 A1
(43) Pub. Date: Dec. 16, 2010

(54) Systems and Methods for Implanting Medical Devices

(76) Inventors: Robert E. Lund, Minnetonka, MN (US); John Jason Buysman, Minnetonka, MN (US)

Correspondence Address: AMS RESEARCH CORPORATION, 10700 BREN ROAD WEST, MINNETONKA, MN, 55343, US

(21) Appl. No.: 12/226,293

(22) PCT Filed: Mar. 20, 2007
(86) PCT No.: PCT/US2007/006889

§ 371 Date: Aug. 24, 2010

Related U.S. Application Data
(60) Provisional application No. 60/788,979, filed on Apr. 4, 2006.

(51) Int. Cl. A61B 18/00

(52) U.S. Cl. 606/129

(57) ABSTRACT

Methods and instruments forming a tissue pathway between the first and second skin incisions to draw a permanent or temporary elongated medical device or implant, e.g., a medical electrical lead, through the tissue pathway are disclosed. A tunneling instrument comprises an elongated sleeve and a needle that are mounted together to be used as the tunneling instrument to form the tissue pathway and separated so that the sleeve may be employed to engage the lead connector to pull the medical electrical lead through the tissue pathway.

### RELATED APPLICATION

This application claims priority to U.S. Provisional Application Ser. No. 60/788,979 filed Apr. 4, 2006, the entire content of which is incorporated herein by reference.

### TECHNICAL FIELD

The present invention pertains to improved methods and apparatus to route a permanent or temporary elongated medical device or implant between first and second skin incisions that may be employed in forming a tissue pathway between the first and second skin incisions and drawing a medical electrical lead through the tissue pathway to couple the lead connector with an implantable pulse generator (IPG) or an extension wire from an IPG implanted a further remote site in the body.

### BACKGROUND

As set forth in U.S. Pat. No. 6,862,480 urinary incontinence is a significant clinical problem and a major source of disability and dependency. The most frequently occurring types of urinary incontinence are stress incontinence, urge incontinence, overflow incontinence, reflex incontinence, and mixed incontinence characterized by involuntary loss of urine, beyond the individual's control, due to the loss or diminution of the ability to maintain the urethral sphincter closed as the bladder fills with urine. Muscles involved in controlling the urinary flow include primarily the urethral sphincter and the levator ani, with the cooperation of fibromuscular extensions along the urethra and other muscles in the general region of the pelvic diaphragm.

Male or female stress urinary incontinence (SUI) occurs and results from weakness or inability of pelvic muscles to hold back urinary flow from the bladder when abdominal pressure increases due to common physical or emotional stress events, such as coughing, laughing or mild physical exertion. Stress incontinence is typically associated with either or both of urethral hypermobility and intrinsic sphincter deficiency. Urethral hypermobility is characterized by weakness of or injury to pelvic floor muscles that causes the bladder to descend during abdominal straining or pressure, allowing urine to leak out of the bladder. Intrinsic sphincter deficiency is characterized by the inability of the urethral musculature to completely dose the urethra or keep it closed during stress.

In urge incontinence, a sudden, urgent need to pass urine causes involuntary urination, before the patient can get to a toilet. The condition may be caused by damage to nerve pathways from the brain to the bladder or by psychosomatic factors, leading to involuntary bladder contraction. Overflow incontinence occurs when the bladder is unable to empty normally. Weak bladder muscles, caused e.g. by nerve damage from diabetes, or a blocked urethra, caused e.g. by tumors or urinary stones, are among the more common causes of overflow incontinence. Frequency or urgency involves the need or urge to urinate on an excessively frequent or habitual basis. Some patients exhibit combinations of these types of incontinence that are often called mixed incontinence.

Many options are available to treat incontinence in its various forms, including Kegel exercises, biofeedback, timed voiding or bladder training, medications, pessaries, invasive or minimally invasive surgery, catheterization, and implantation IMDs. Urinary incontinence IMDs are typically implanted in relation to the tissue structure around the urethra including the urethral wall, the bladder neck, bladder suspension ligaments, the urethral sphincter, pelvic ligaments, pelvic floor muscles, fascia, and the like. Urinary incontinence IMDs include urethral tapes or slings that support the urethra, prosthetic sphincter systems that compress the urethra until voiding is initiated, and periurethral or transurethral injection of a mass of biocompatible bulk-enhancing or bulking agent or an inflatable balloon into the tissue structure around the urethra.

Urethral slings are implanted employing minimally invasive surgical instruments and techniques to route the sling through a tissue pathway disposing a central portion of the sling extending around the urethra to provide urethral support. The sling implantation tools disclosed in U.S. Patent Application Publication No. 2005/0043580 have a curvature in a single plane and correspond generally to the BioArc™ SP and SPARC™ single use sling implantation tools sold by America medical Systems, Inc., (Minnetonka, Minn.) in a kit with an elongated urethral sling. In each such sling implantation tool, the needle portion has a proximal straight portion extending from the handle and a distal shaped portion terminating in a needle distal end. The needle portion is sized and shaped so that the distal end may initially be moved through an abdominal incision and advanced posterior to one of the right and left posterior ischiopubic pubic ramus of the pelvic girdle spaced from the bladder to a urethral incision accessing the urethral tissue structure, e.g., a vaginal incision in the region of the vaginal apex of a female patient.

In U.S. Patent Application Publication No. 2002/0165566, a curved needle is provided having a detachable handle for making a similar tissue pathway by extending the needle end through the vaginal incision, through the tissue, and then out an abdominal skin incision. The handle is detached, and the sling end coupled to the needle end extending from the vaginal incision. The needle end extending from the abdominal skin incision is then grasped to pull the sling through the tissue pathway so that the sling end can be detached from the needle end outside the abdominal incision.

In another approach, a neuromodulator or neurostimulator IMD is implanted in a patient's body to electrically stimulate excitable muscle tissue, nerves, and organs in the pelvic region to treat pelvic floor disorders including urinary and fecal incontinence, erectile dysfunction, and pelvic pain. In one approach, nerves controlling external sphincter and bladder functions, e.g., the sacral nerves in the nerve root or at the peripheral sciatic nerve or the pudendal nerve, are stimulated.

According to several known surgical treatment methods, one or more nerve stimulation or neural stimulation electrode supported at the distal end of a neural lead is disposed at a nerve stimulation site. It is typically necessary to employ introducers and stiffening stylets and/or guidewires to position the distal neural stimulation electrode(s) in operative relation to a nerve at the target stimulation site to. The proximal lead connector is coupled to a connector header of an IPG so that the IPG and neural lead comprise the neurostimulator IMD. See for example, U.S. Pat. Nos. 5,569,351, 4,607,639, 4,739,764, 4,771,779, and 6,055,456 regarding electrical stimulation of the sacral nerve to control bladder function. The Interstim® IMD sold by Medtronic Neurological, Columbia Heights, Minn., is one such neurostimulator IMD clinically available for treatment of urge incontinence.

Stimulation of the pudendal nerve employing a neurostimulator IMD as an alternative to sacral nerve stimulation has long been proposed. Electrical stimulation delivered by an intravaginal or a perineal surface electrode has been shown to inhibit premature and inappropriate detrusor contractions. The mechanism for such effects appears to derive from the electrical stimulation of pudendal nerve afferents (sensory receptors or sensory nerve fibers). Input into the pudendal afferent system inhibits a parasympathetic reflex loop consisting of bladder wall afferents (sensory reflexes) and efferents (motor reflexes). This parasympathetic loop normally senses a distension of the bladder via the afferent limb and responds by sending an efferent signal to contract the bladder. Although such stimulation has shown therapeutic effects, electrode placement and on-going stimulation do not lend themselves easily to chronic stimulation.

In another approach, a muscle tissue stimulator IMD is implanted in a patient's body to directly electrically excitable muscle tissue of a sphincter, e.g., tissue structure around the urethra. For convenience, the expressions “tissue stimulator” and “tissue stimulation” may be employed herein to characterize IMDs comprising medical electrical leads and IPGs and the stimulation applied to tissue structures of the abdominopelvic or simply pelvic region to enervate to cause muscle tissues to contract. Exemplary muscle tissue stimulator IMDs (or simply tissue stimulators) for treatment of urinary incontinence and neurogenic bladder dysfunction are disclosed, for example, in Biocontrol Medical Ltd. U.S. Pat. Nos. 6,354,991, 6,652,449, 6,712,772, and 6,862,480 and U.S. Patent Application Publication 2005/0216069. The tissue stimulators disclosed in the Biocontrol Medical patents for treatment of both urinary stress incontinence and urge incontinence comprise a control unit or IPG and one or more medical electrical leads bearing one or more sensing/stimulation electrode and one or more physiologic sensor adapted to be implanted in selected sites of a patient's body. The sensing/stimulation electrode(s) is preferably implanted in the pelvic region of a patient so as to be in electrical contact with body tissue including one or more of the muscles that relax and contract in regulating urine flow from the bladder. The control unit is preferably implanted under the skin of the abdomen or genital region, and receives signals from the electrodes and/or from the sensors. Motion and/or pressure signals detected by the physiologic sensor(s) and/or electromyogram (EMG) signals appearing across the sensing/stimulation electrodes are conveyed to and analyzed by the control unit operating system in order to distinguish between signals indicative of urge incontinence and those indicative of stress incontinence. A particular pressure sensor design is disclosed in the above-referenced '772 patent. When impending stress incontinence is detected, the control unit generates and provides an electrical stimulation therapy having stimulation parameters configured to treat stress incontinence through the electrodes to the tissue. Similarly, urge incontinence is treated with intermittent electrical stimulation having stimulation parameters configured to treat urge incontinence.

In various configurations, the tissue stimulators disclosed in the above-referenced Biocontrol Medical patents may be used alternatively or additionally to treat fecal incontinence, interstitial cystitis, urine retention, or other sources of pelvic dysfunction, pain or discomfort, by suitable modifications to the IMD.

The control unit or IPG disclosed in the above-referenced Biocontrol Medical patents is preferably implanted under the skin of the abdomen or genital region, the stimulation/sense electrodes are preferably implanted in the pelvic region so as to be in electrical contact with one or more of the muscles that regulate urine flow from the bladder, e.g., the urethral sphincter and the levator ani, and the mechanical sensors are preferably implanted on, in or in the vicinity of the bladder. The stimulation/sense electrodes are described as flexible wire, intramuscular-type, electrodes, about 1-5 mm long and 50-100 microns in diameter, and may be formed in the shape of a spiral or hook, so that the shape facilitates fixation in tissue. The mechanical sensors supported on an sensor lead comprise one or more pressure, force, motion or acceleration sensor, or an ultrasound transducer, that generate signals responsive to motion, to intravesical or abdominal pressure, or to urine volume in the bladder, and are thus indicative of possible imminent incontinence.

Sensing circuitry in the control unit or IPG receives and processes electromyographic (EMG) signals sensed across the electrodes and the mechanical sensor output signal to distinguish between EMG signals indicative of urge incontinence, EMG signals indicative of stress incontinence, and EMG signals that are not due to incontinence. Electrical stimulation pulses having stimulation parameters tailored to inhibit urge incontinence are generated by the IPG and delivered across the electrodes when the sensed signals are indicative of impending urge incontinence. Similarly, electrical stimulation pulses having stimulation parameters tailored to inhibit stress incontinence are generated by the IPG and delivered across the electrodes when the sensed signals are indicative of impending stress incontinence.

Certain implantation methods for implanting the tissue stimulation IMD in the body of a female patient are described in the '651 and '480 patents. It is suggested that similar methods would be employed in the implantation of the IMD in a male patient.

In one implantation method shown in FIGS. 2A-2G of the '480 patent, a subcutaneous surgical pocket is made to receive the IPG approximately 1 cm cephalad to the pubic bone. A vaginal mucosa incision is made at a site approximately 0.5-1 cm anterior and lateral to the urethral meatus. A 5 French, splittable short introducer is inserted into the vaginal mucosa incision adjacent to the lead and advanced with care slightly medially, i.e., towards the urethra, about 2.5 cm, to a site 0.5-1 cm lateral to the urethral wall. The electrode and fixation mechanism (a spiral helix or hook) are advanced through the splittable introducer lumen of the introducer extending from the vaginal mucosa incision to the stimulation and fixation site proximate the urethral sphincter. The introducer sleeve is split apart to withdraw it over the lead body after the stimulation electrode is properly positioned. The stimulation lead body is sutured to the subcutaneous tissue to secure it from movement.

A subcutaneous tunnel or pathway is tunneled between the pocket and the vaginal mucosa incision, and the lead body is extended through the pathway to dispose a distal portion of the lead outside the vaginal mucosa incision. In one embodiment of the '480 patent, the tunneling of the lead body between the vaginal mucosa incision and the suprapubic incision is effected by subcutaneously tunneling a 12 Fr introducer from the either incision to the other incision and passing the lead, distal end first, from the suprapubic incision through the introducer lumen to the vaginal mucosa incision and then removing the introducer over the lead body. The exposed distal portion of the lead body is retracted subcutaneously, and the vaginal mucosa incision is closed.

To implant the sensor lead, an 8 French introducer is inserted through the pocket incision, between the fascia and muscle tissue, and advanced into the retropubic space. The sensor lead bearing a distal pressure or electrical sensor is stiffened by a stiffening stylet and the lead body is advanced through the introducer to dispose the sensor at a desired position, e.g., in the retropubic space or between fascia and muscle. The sensor lead body is also sutured to the fascia, the stylet is withdrawn, and the introducer is removed. The sensor lead and stimulation lead connectors are coupled to the IPG. The IPG is disposed in the pocket and the pocket is closed after testing the IMD to ascertain that all connections are secure and that sensing and stimulation can be reliably provided.

In NDI Medical U.S. Patent Application Publication No. 2006/0004429, a similar surgical technique is employed in the implantation of an IPG and medical electrical lead. In the disclosed approach, the site for the needle puncture to advance the lead electrodes to the stimulation site is located midline or near-midline, near the inferior border of the pubic symphysis aiming toward the clitoris (or the base of the penis in males). The incision site for forming the subcutaneous pocket to receive IPG comprises a lateral 2 cm incision (see FIG. 15), which is located two finger-breaths medial to the anterior iliac spine and made in the direction of the dermatomal skin line.

A subcutaneous tunnel is formed for routing the medical electrical lead body to the IPG to be implanted in the subcutaneous pocket. The size of the needle puncture is increased using a skin knife, and an elongated tunneling tool 40 (shown in FIG. 2) is passed through the pocket incision site (see FIG. 16) toward and through the needle puncture site. The tunneling tool desirably includes a removable blunt tip that is present during tunneling, but that is removed once passage through the distant incision site occurs. In an alternative approach, the tunneling tool comprises an elongated stylet and sheath, and the stylet and sheath are advanced from the needle puncture site toward the pocket incision. Following removal of the blunt tip or the stylet, the medical electrical lead can be passed through the open lumen of the tunneling tool or sheath to dispose the lead connector proximate the pocket incision site after the tunneling tool is retracted over the lead body.

In the procedures described above, the lead body is pushed, connector-end first, through the lumen of the sheath or tunneling tool from one incision site to another incision site, and the sheath or tunneling tool is retracted over the lead body to enable connection of the lead connector with the IPG connector. It is necessary to provide a tool or sheath lumen that is larger than the largest diameter features of the medical electrical lead, which typically comprise the proximal lead connector or the distal electrode fixation mechanism so that the lead can be readily pushed through the lumen without interference. The larger lumen diameter dictates the outer diameter of the tunneling tool or sheath, resulting in a tissue pathway that exceeds the lead body diameter.

Tunneling tools employed in the subcutaneous routing of other medical electrical leads or catheters typically employ a tunneling tool having a distal tip configured to receive or engage a lead connector mechanism or a catheter end to pull the lead through a tissue pathway created by the tunneling tool.

In U.S. Pat. No. 4,832,687, a catheter is attached to the distal end of the tunneling needle (after removal of a bullet-shaped, tissue separating tip) by interference fitting the threaded needle distal end into the catheter lumen so that the catheter can be pulled back through the tissue pathway created by the tunneling needle. See also U.S. Pat. Nos. 4,574,806, 5,234,438, 5,306,240, 6,565,594, and 7,128,734.

Tunneling tools for creating a tissue pathway to route cardiac pacing and cardioversion-defibrillation leads are well known in the art and include U.S. Pat. Nos. 4,010,757, 5,782,841, 5,871,528, 6,605,094, and 7,018,384, as well as the Traverser™ IS-1 lead tunneling tool sold by Pressure Products, Inc. Currently, bipolar medical electrical leads are fabricated with lead connectors that are shaped and dimensioned to conform to ISO standards so that a variety of leads may be inserted into connector bores of IPGs that conform to the same standard. The low profile connector “IS-1” standard (ISO 5841-3:1992(E)) is one such standard for bipolar in-line and unipolar lead connectors and IPG bores and connectors. Certain of the tunneling tools have bores or receptacles that are shaped to conform to the IS-1 standard or other standards.

The Traverser™ IS-1 lead tunneling tool has an enlarged diameter tool distal end having an external taper shaped to dilate the pathway created by the tool proximal end as it is pushed from the lead distal end through subcutaneous tissues to create the tissue pathway from a lead implantation skin incision to an IPG pocket incision. An axial bore extends proximally into the tunneling tool from the tool distal end and toward the tool proximal end. The bore length corresponds to the IS-1 connector length, and the bore diameter is smaller than the IS-1 sealing ring diameters.

In the '384 patent, the lead connector is fitted through a side-loading slot at the distal end of a tunneler into a cradle shaped to receive the connector so that the lead can be drawn through the pathway as the tunneler is retracted. The tunneler has a removable bullet-shaped tip fitted into the cradle to create the tunnel. The IS-1 lead connector is not advanced proximally into an instrument lumen, but a slidable cover can be slid distally over the lead connector to the needle distal end to isolate the lead connector from contact with body fluids and tissue.

Despite these improvements, it would be desirable to provide a simplified and inexpensive manner of drawing a medical electrical lead (or other elongated medical device or implant) through a tissue pathway, particularly drawing a medical electrical lead between a vaginal mucosa incision and a suprapubic incision.

### SUMMARY

In accordance with the present invention, a tunneling instrument comprises an elongated sleeve and a needle that are mounted together to be used as the tunneling instrument to form the tissue pathway and separated so that the sleeve may be employed to engage the lead connector to pull the medical electrical lead through the tissue pathway.

A tunneling instrument and method for passing a permanent or temporary elongated medical device or implant, e.g., a medical electrical lead, subcutaneously between a first and a second skin incision, comprises an elongated sleeve and a needle that are mounted together to be used as the tunneling instrument or tool to form a tissue pathway and separated so that the sleeve may be employed to engage the lead connector to pull the medical electrical lead through the tissue pathway. The elongated sleeve may comprise a sleeve body extending from a sleeve first end and a sleeve second end through a sleeve body length sufficient to extend from the first and second skin incisions, the sleeve body enclosing a sleeve lumen having a sleeve lumen diameter. The elongated needle may comprise a needle shaft extending between a needle first end and a needle second end having a needle shaft diameter and a shaft length sufficient to extend from the first and second skin incisions. The sleeve lumen diameter is sized to the needle shaft diameter to enable insertion of the needle shaft through the sleeve lumen with the needle second end in proximity to the sleeve second end to form the tunneling instrument or tool adapted to form a subcutaneous tissue pathway between the first and second incision and withdrawal of the needle shaft from the sleeve lumen. The sleeve lumen diameter is also sized to the lead connector diameter at least proximate the sleeve second end to provide frictional engagement of the lead connector against the sleeve body upon insertion of the lead connector into the sleeve lumen so that the sleeve and lead body may be pulled through the tissue pathway to dispose the lead connector outside the first skin incision.

In one preferred embodiment, a sleeve lumen window to the sleeve lumen is formed across the sleeve second end to enable axial advancement of the lead connector through the sleeve lumen window to frictionally engage the sleeve body.

In another preferred embodiment, a sleeve lumen window to the sleeve lumen is formed as a slot through the sleeve body extending from substantially the sleeve second end toward the sleeve first end to enable lateral insertion of the lead connector through, the slot and axial advancement of the lead connector toward the sleeve first end to frictionally engage the sleeve body.

In still another preferred embodiment, a handle is permanently or temporarily attached to the needle shaft at the needle first end and is adapted to be grasped to advance the tunneling instrument through tissue to form the tissue pathway. The sleeve first end abuts the handle when the needle shaft is inserted through the sleeve lumen to fix the sleeve in place during advancement of the tunneling instrument through body tissue to create the tissue pathway.

In further preferred embodiments, the sleeve body at the sleeve second end may be shaped to have a sleeve lumen diameter that varies or narrows at one or more point to facilitate frictional engagement with the lead connector.

In still further preferred embodiments, the sleeve lumen surface or sleeve body material proximate the sleeve second end may be treated or coated to enhance frictional engagement with the lead connector.

Typically, the lead connector is formed having a resilient sealing ring having a sealing ring diameter exceeding the sleeve lumen diameter, whereby frictional engagement of the lead connector with the sleeve body is attained by compression of the resilient sealing ring within the sleeve lumen.

One aspect of the invention involves use of the tunneling instruments and methods of the present invention in the implantation of tissue stimulators for delivering tissue stimulation through stimulation/sense electrode(s) to selected sites in the pelvic region to treat selected pelvic disorders. Such selected sites include the urethral sphincter and optionally the levator ani, to deliver electrical stimulation to treat urinary incontinence.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a plan view of a bipolar medical electrical lead and stiffening stylet, the lead having a lead connector at the lead proximal end and a pair of stimulation/sense electrodes and fixation mechanism at the lead distal end;

FIG. 2 is a side view of a needle having a curved needle shaft coupled to a handle at the needle first end and extending through a needle shaft length to a needle second end;

FIG. 3 is an end view of the needle of FIG. 2;

FIG. 4 is a top view of the needle of FIG. 2 and an elongated sleeve having a sleeve lumen extending from a sleeve first end to a sleeve second end, the needle second end positioned to be inserted through a sleeve lumen opening at the sleeve first end and extended through the sleeve lumen;

FIG. 5 is a schematic depiction of the needle shaft extended through the sleeve lumen to abut the sleeve first end against the handle and to extend the needle shaft second from the sleeve lumen opening at the sleeve second end, thereby forming a tunneling instrument of the present invention;

FIG. 6 is a schematic illustration in partial cross-section of the needle, sleeve, and lead connector juxtaposed to identify their relative diameters;

FIG. 7 is a schematic illustration of the extension of the lead body and lead connector from a second incision and the use of the tunneling instrument to form a tissue pathway from a first incision to a second incision;

FIG. 8 is a schematic illustration of the insertion of the lead connector extending from the second incision into the sleeve lumen opening at the sleeve second end following retraction of the needle shaft from the sleeve lumen;

FIG. 9 is a schematic illustration of the lead connector extending from the second incision inserted into the sleeve lumen opening at the sleeve second end;

FIG. 10 is a schematic illustration of the stimulation/sense electrodes and fixation helix disposed at a stimulation site and the retraction of the sleeve from the first incision to draw the medical electrical lead into the second incision and through the tissue pathway;

FIG. 11 is a schematic illustration of the stimulation/sense electrodes and fixation helix disposed at a stimulation site and the lead connector drawn out of the first incision with the lead body extending through the tissue pathway;

FIG. 12 is a schematic illustration of the extension of the lead body and lead connector from the second incision and the use of the tunneling instrument to form a tissue pathway from a first incision to a second incision, wherein the sleeve has a sleeve lumen window to the sleeve lumen formed as a slot through the sleeve body extending from substantially the sleeve second end toward the sleeve first end;

FIG. 13 is a schematic illustration of the extension of the lead body and lead connector from the second incision after retraction of the needle shaft from the sleeve lumen of the sleeve depicted in FIG. 12;

FIG. 14 is a schematic illustration of the extension of the lead body and lead connector from a second incision and the lateral insertion of the lead connector through the sleeve lumen window into the sleeve lumen of the sleeve depicted in FIGS. 12 and 13;

FIG. 15 is a schematic illustration of the stimulation/sense electrodes and fixation helix disposed at a stimulation site, the lead connector advanced into the sleeve lumen of the sleeve depicted in FIGS. 12-14 toward the sleeve first end, and the retraction of the sleeve from the first incision to draw the medical electrical lead into the second incision and through the tissue pathway;

FIG. 16 is a schematic depiction in partial cross-section of a sleeve segment adapted to have internal features receiving a lead connector and enhancing engagement with the lead connector;

FIG. 17 is a schematic depiction in partial cross-section of the lead connector inserted into the sleeve segment of the sleeve body depicted in FIG. 16 with the internal features engaging the lead connector;

FIG. 18 is a schematic depiction in partial cross-section of a sleeve segment adapted to have further internal features receiving a lead connector and enhancing engagement with the lead connector; and

FIG. 19 is a schematic depiction in partial cross-section of the lead connector inserted into the sleeve segment of the sleeve body depicted in FIG. 18 with the internal features engaging the lead connector;

The figures are not necessarily to scale.

### DETAILED DESCRIPTION OF PREFERRED EMBODIMENTS

Various features and aspects of the present invention may be practiced separately or in combination and may find application in the positioning and fixation of medical electrical leads in various parts of the pelvic region to treat various pelvic disorders of male and female patients. In either male or female patients, the same or similar tunneling instruments and procedures may be employed in subcutaneously routing lead bodies of medical electrical leads.

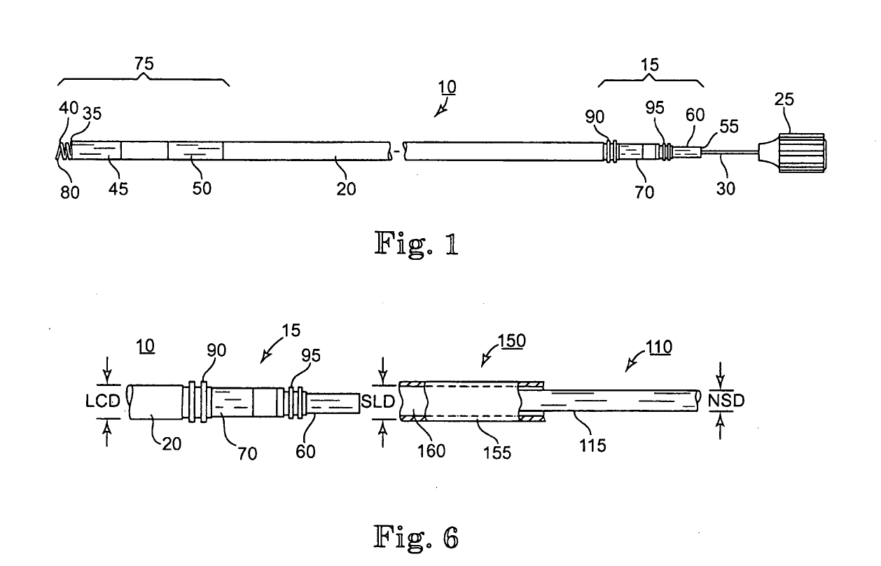

An exemplary medical electrical lead that may be implanted employing the implantation tools, kits, systems, and methods of the present invention and coupled to a tissue stimulation IPG is depicted in FIG. 1. Exemplary tunneling instruments or tools used in and steps of implanting an exemplary medical electrical lead and coupling the lead connector to a tissue stimulation IPG are depicted in FIGS. 2-5. Particular steps in the implantation procedure and variations of the tunneling instruments, kits, systems, and methods of the present invention are depicted in the remaining figures.

In accordance with one aspect of the present invention, conventional multi-polar and unipolar medical electrical leads, e.g., the bipolar medical electrical lead 10 depicted in FIG. 1, may be implanted employing the implantation tools, kits, systems, and methods of the present invention and coupled to a compatible tissue stimulation IPG. The medical electrical lead 10 is operable, when coupled to an IPG, to transmit EMG signals to the IPG sense amplifier (if present in the IPG), and to deliver the stimulation from an IPG output circuit to a stimulation site of the patient's body, particularly the region of the urethra, in the treatment of incontinence. It will be understood that the term “stimulation/sense electrode” used herein embraces an electrode that can be employed to deliver neurostimulation from such an IPG output circuit and to conduct EMG signals to an IPG sense amplifier, if present in the IPG circuitry.

The bipolar medical electrical lead 10 comprises an elongated lead body 20 extending from a proximal lead connector 15 comprising spaced apart lead connector elements 60 and 70 to spaced apart, stimulation/sense electrodes 45 and 50, respectively, for bipolar stimulation of tissue at the stimulation site. The lead body 20 is formed of an electrically insulating sheath encasing electrical conductors that extend from the distal electrodes 45 and 50 to connector elements 60 and 70, respectively, of the lead connector 15.

The lead connector assembly 15 further comprises resilient sealing rings 95 between lead connector pin 60 and ring 70 and further resilient sealing rings 90 distal to connector ring 70. The resilient sealing rings 95, the lead connector pin 60, the lead connector ring 70, and the further resilient sealing rings 90 may conform in size and shape to a conventional bipolar, in-line connector assembly, e.g., conforming to the IS-1 bipolar lead standard.

The distal stimulation/sense electrode or tip electrode 45 extending to the lead body distal end 35 is coupled to a spiral fixation helix 40. The spiral fixation helix 40 preferably has a constant pitch and diameter and terminates in a point 80. The spiral fixation helix 40 may be mechanically and electrically coupled to the tip electrode 45 or separated from the tip electrode 45. For convenience, a distal lead segment 75 is designated embracing the spiral fixation helix 40, the tip and ring stimulation/sense electrodes 45 and 50 and a portion of the lead body 20,

A lead body lumen 55 extends axially along the lead body 20 from a proximal lumen opening in the connector pin 60 into the distal lead segment 75. A stylet wire 30 coupled to a stylet handle 25 is inserted into the lead body lumen 55 to stiffen the lead body 25 while the distal electrodes 45 are positioned at a stimulation site. The lead connector 15 may be rotated with the stylet wire 55 in place to rotate the lead body 20 and screw the helix 40 into tissue at the stimulation site.

A tunneling instrument or tool 100 for passing the medical electrical lead of FIG. 1 subcutaneously between a first and a second skin incision, comprises an elongated needle 110 and a sleeve 150 that are mounted together as shown in FIGS. 2-5 to form the subcutaneous tissue pathway. The elongated needle 110 is then separated from the sleeve 150 so that it may be employed to engage the lead connector 15 to pull the medical electrical lead 10 through the tissue pathway.

The elongated needle 110 may comprise a needle shaft 115 extending between a needle first end 125 and a needle second end 130 having a needle shaft diameter NSD and a shaft length sufficient to extend from a first skin incision to a second skin incision. A handle 120 is permanently or temporarily attached to the needle shaft 115 at the needle first end 125. The needle second end 130 is shaped to provide blunt dissection and tissue separation as it is advanced into tissue. It will be understood that the needle first and second ends 125 and 130 may have the same shape, and that the handle 120 is formed with needle end receptacle for detachably receiving and engaging either needle end.

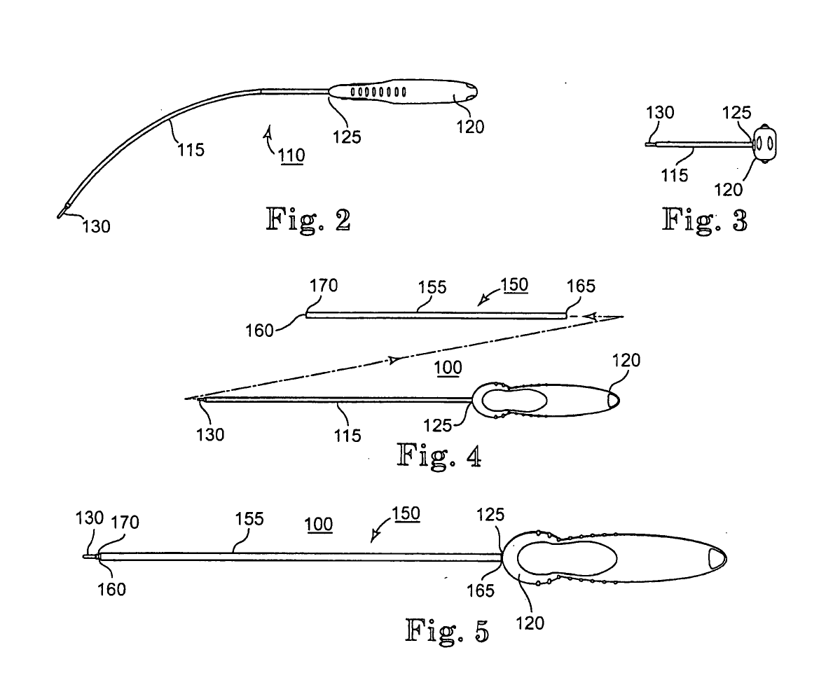

The needle shaft can be curved in a single plane as depicted in FIGS. 2 and 3 or can be curved in three dimensions. It will also be understood that the needle shaft 115 may be formed of a malleable material so the surgeon can customize the needle shaft shape and curvature to accommodate the surgeon's preferences and the physiology of the particular patient.

The elongated sleeve 150 shown in FIGS. 4 and 5 may comprise a sleeve body 155 that is preferably flexible and transparent and extends from a sleeve first end 165 to a sleeve second end 170 through a sleeve body length sufficient to extend from the first and second skin incisions. The sleeve body 155 encloses a sleeve lumen 160 having a sleeve lumen diameter SLD and extending between sleeve lumen openings at the sleeve first and second ends 165 and 170. The sleeve lumen diameter SLD is sized to the needle shaft diameter NSD to enable insertion of the needle shaft 115 through the sleeve lumen 160 as shown in FIG. 4. The tunneling instrument or tool 100 so formed with the needle second end 130 in proximity to the sleeve second end 170 is adapted to create a subcutaneous tissue pathway between the first and second incisions by manipulation of the handle 120. The sleeve first end 165 abuts the handle 120 as shown in FIG. 5 when the needle shaft 115 is fully inserted through the sleeve lumen 160 to fix the sleeve 150 in place during advancement of the tunneling instrument 100 through body tissue to create the tissue pathway.

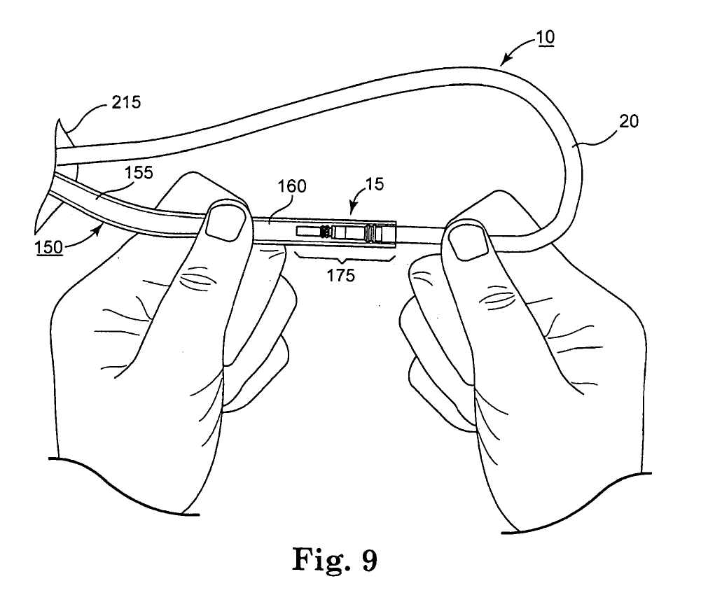

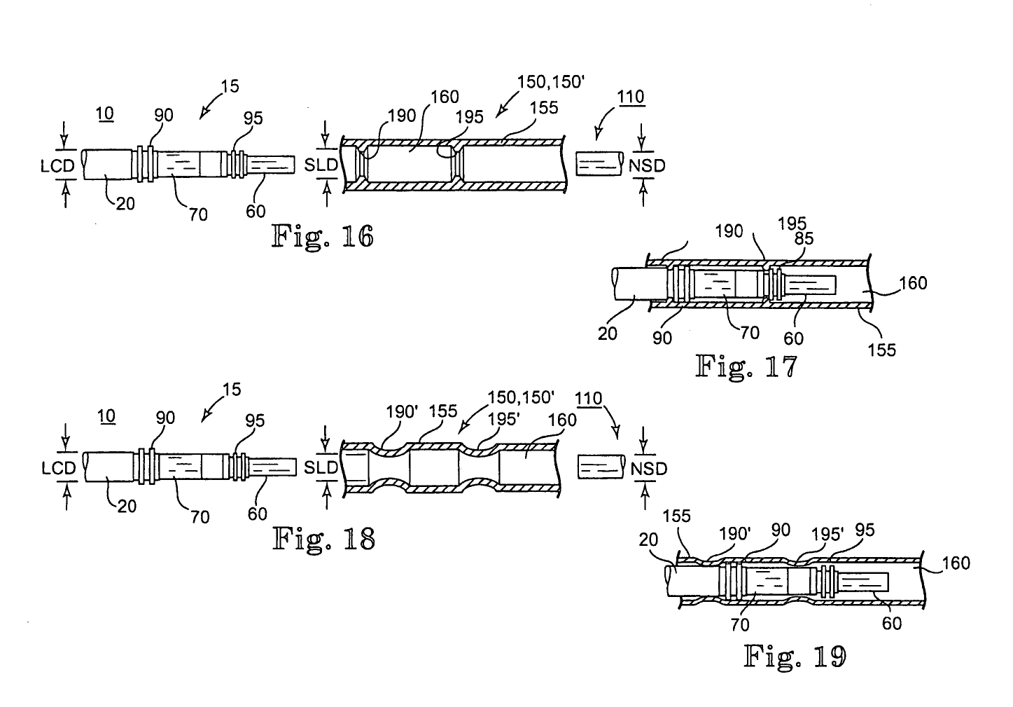

Referring to FIG. 6, the needle shaft 115 has a needle shaft diameter NSD that is sufficiently smaller than the sleeve lumen diameter SLD so that the needle shaft 115 may be readily withdrawn from the sleeve lumen 160 after the tissue pathway from the first incision to the second incision is created. The sleeve lumen diameter SLD of sleeve lumen 160 is sized to the lead connector diameter LCD at least within a connector receiving chamber 175 (shown in FIG. 9) proximate the sleeve second end 170 to provide frictional engagement of the lead connector 15 against the sleeve body 155 upon insertion of the lead connector 15 into the sleeve lumen 160. Typically, the resilient sealing rings 90 and 95 having a sealing ring diameter exceeding the sleeve lumen diameter SLD at least proximate the sleeve second end 170, whereby frictional engagement of the lead connector 15 with the sleeve body 155 within connector receiving chamber 175 is attained by compression of the resilient sealing rings 90 and/or 95 within the sleeve lumen 160 against the sleeve body 155. The sleeve lumen diameter SLD at least proximate the sleeve second end 170 and within the length of a connector receiving chamber 175 may be a constant or periodic diameter as illustrated in FIGS. 16-19 described further below.

The interior surface of the sleeve body 155 at least proximate the sleeve second end 170 within the connector receiving chamber 175 may also be treated with a biocompatible surface coating that enhances frictional engagement with the lead connector 15 when it is inserted into the connector receiving chamber 175. Alternatively, at least that portion of the sleeve body may be formed of a biocompatible material presenting a tacky surface that enhances frictional engagement with the lead connector 15 when it is inserted into the connector receiving chamber 175.

The frictional engagement facilitates the routing of the lead body 20 through the tissue pathway as the sleeve 150 is pulled through the tissue pathway and out of the first skin incision to dispose the lead connector 15 outside the first skin incision. The lead connector 15 may then be withdrawn from the connector receiving chamber 175 and coupled to an IPG connector of an IPG to be implanted in a pocket formed at the first skin incision 210. Alternatively, the lead connector 15 may be coupled to a lead extension that is itself tunneled from the first skin incision 210 to a third skin incision (not shown) in the manner of the present invention or otherwise to be coupled to an IPG connector of an IPG to be implanted in a pocket formed at the third skin incision.

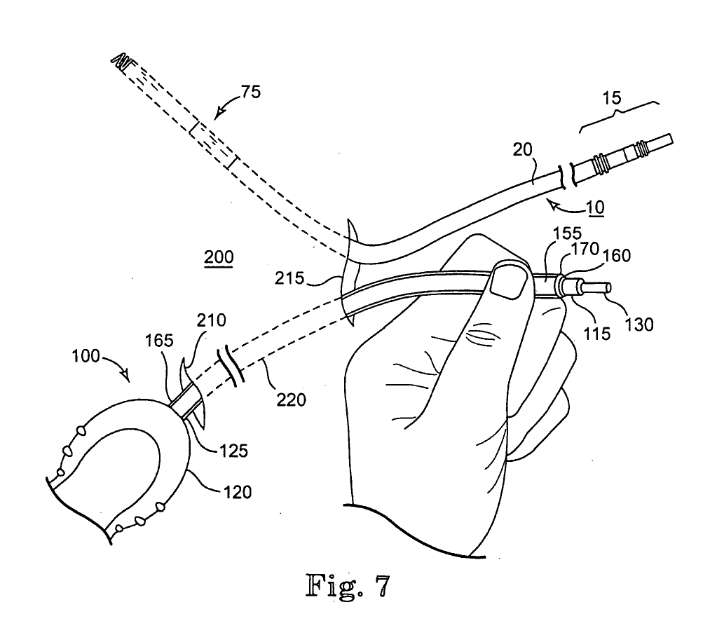

In one preferred embodiment of the tunneling instrument 100 depicted in FIGS. 7-11, a sleeve lumen window to the sleeve lumen 160 is formed transversely across the sleeve second end 170 to enable advancement of the lead connector 15 through the sleeve lumen window into the connector receiving chamber 175 to frictionally engage the sleeve body 155. One exemplary method of forming the tissue pathway between the first skin incision 210 the and second skin incision 215 and passing the medical electrical lead 10 through the tissue pathway is also depicted in FIGS. 7-11. In this illustrated method, the first skin incision 210 may be an abdominal skin incision through the skin 200 in an abdominal region of the patient's body, and the second skin incision 215 may be a urethral skin incision through the skin of a male or female patient. The urethral skin incision is made at a point where a relatively short distal portion of the lead body 20 may be advantageously advanced employing an introducer and stylet to dispose the distal fixation helix and stimulation/sense electrodes at a urethral stimulation site. The second skin incision 215 may be made to access and expose a section of the tissue structure around the urethra between the urethral orifice and the vaginal orifice that includes the target tissue structure for stimulation.

Thus, the initial steps in the method comprise making the incisions and implanting the distal portion of the lead body to dispose the stimulation/sense electrodes 45 and 50 of the distal lead segment 75 in or adjacent urethral tissue structure of the urethra of a patient leaving the relatively longer proximal portion of the lead body outside the second skin incision 215 as schematically illustrated in FIG. 7. A suture may be passed through tissue exposed by the second or urethral skin incision and extended around the lead body 20 to stabilize the lead body 20 and inhibit dislodgement of the stimulation/sense electrodes from the stimulation site.

As also illustrated in FIG. 7, the handle 120 of the tunneling instrument 100 is then manipulated to insert and advance the second ends of the assembled sleeve 150 and needle shaft 115 into the first or abdominal skin incision 210 and through subcutaneous tissue to the second or urethral skin incision 215, thereby creating the tissue pathway 220. The needle and sleeve second ends 130 and 170 are disposed outside of the urethral skin incision 215.

It will be understood that if the handle 120 can be attached to either needle end 125 or 130 of the needle shaft 115, then the procedure may be reversed. The handle 120 can be attached to the needle end 125 as depicted in FIGS. 2-5 and manipulated to advance the needle shaft 115 and sleeve 150 in the opposite direction from the second or urethral skin incision 215 to the first or abdominal skin incision 210 to form the tissue pathway 220. Then, handle 120 can be detached from needle end 125 and optionally attached to the needle end 130. In either case, the needle shaft 115 remains in the sheath lumen 160 as shown in FIG. 7 at this point in the procedure.

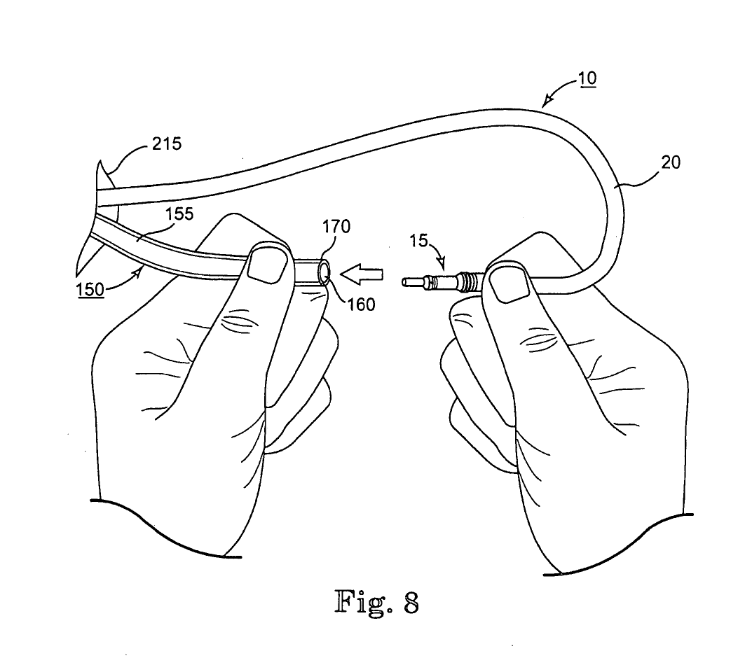

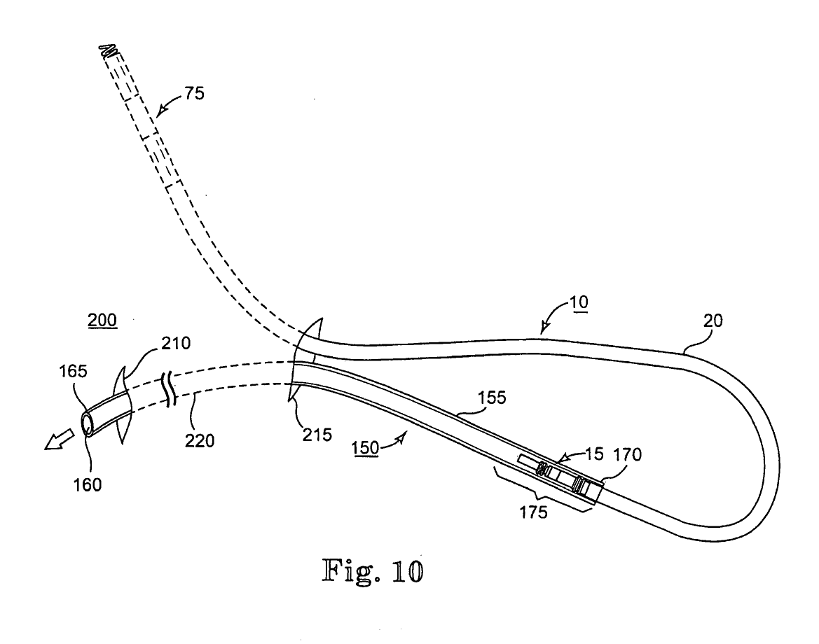

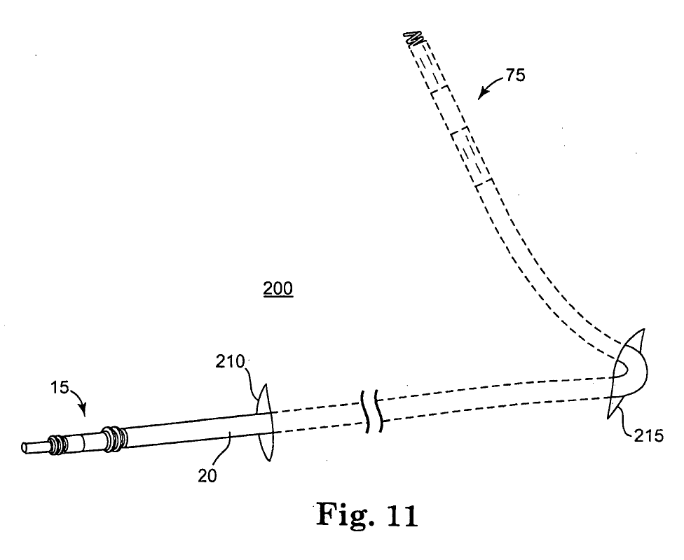

The needle shaft 115 is then withdrawn from the sheath lumen 160, and the lead connector 15 is grasped to insert it axially through the lumen window into the connector receiving chamber 175 of sleeve lumen 160 as shown in FIGS. 8 and 9. The sleeve first end 165 extending from the first or abdominal skin incision 210 is then grasped in FIG. 10 to pull it and the attached proximal portion of the lead body 20 through the tissue pathway 220. The lead connector 15 is thereby drawn out of the sleeve lumen 160 as shown in FIG. 11.

The implantation of a tissue stimulator comprising a medical electrical lead implanted in accordance with the above steps may further comprise forming a subcutaneous tissue pocket proximate the first or abdominal skin incision 210, coupling a tissue stimulation IPG to the lead connector 15, inserting the IPG into the pocket, and closing the incision 210 over the pocket.

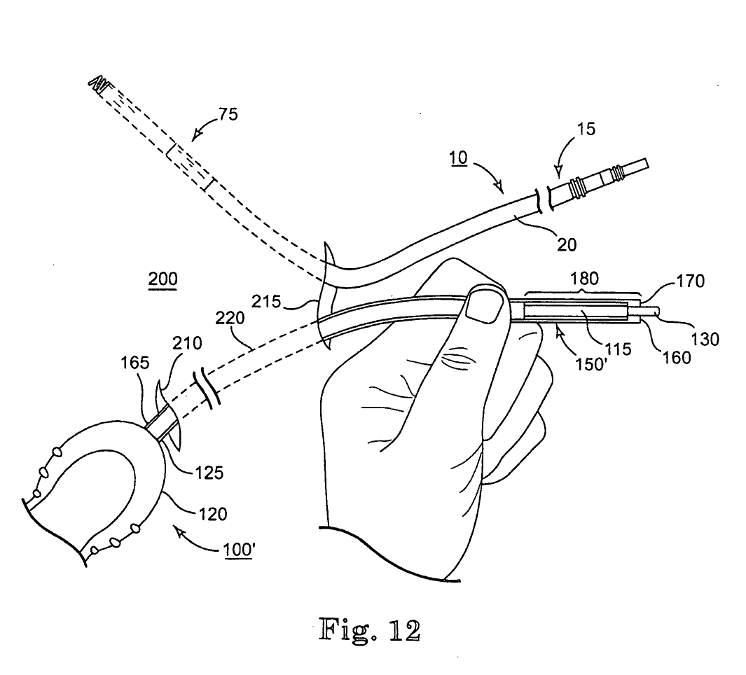

In another preferred embodiment depicted in FIGS. 12-16, a modified tunneling instrument is employed to form the tissue pathway 220 between the first and second skin incisions 210 and 215 and to draw the proximal portion of the lead body 20 through the tissue pathway 220 disposing the lead connector 15 outside the first skin incision 210. The tunneling instrument 100′ is identical to the tunneling instrument 100 except that the modified sleeve 150′ is substituted for the sleeve 150. The modified sleeve 150′ incorporates a sleeve lumen window 180, e.g., a slot through the sleeve body 155, into the sleeve lumen 160. The sleeve lumen window 180 extends from substantially the sleeve second end 170 toward the sleeve first end 165 and has a length accommodating the length of the lead conductor 15. The connector receiving chamber 175 extends from the sleeve lumen window 180 toward the sleeve first end 165.

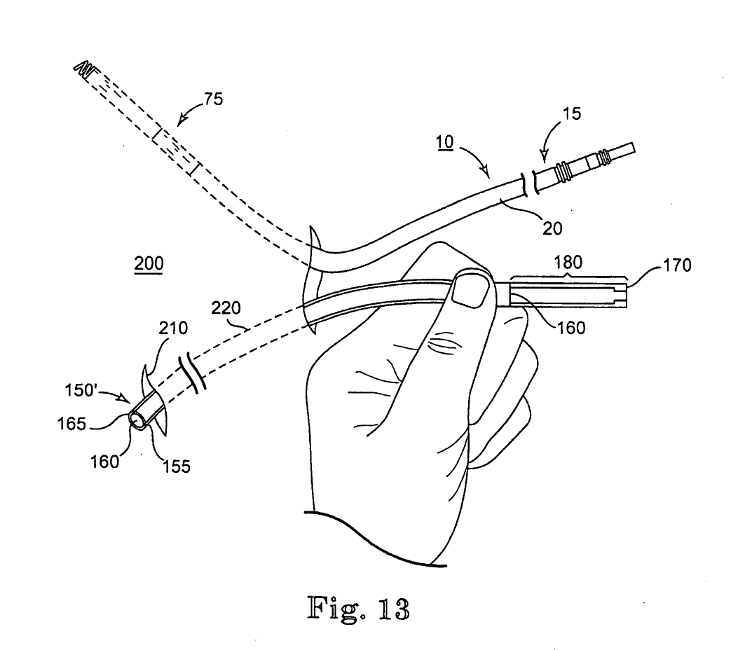

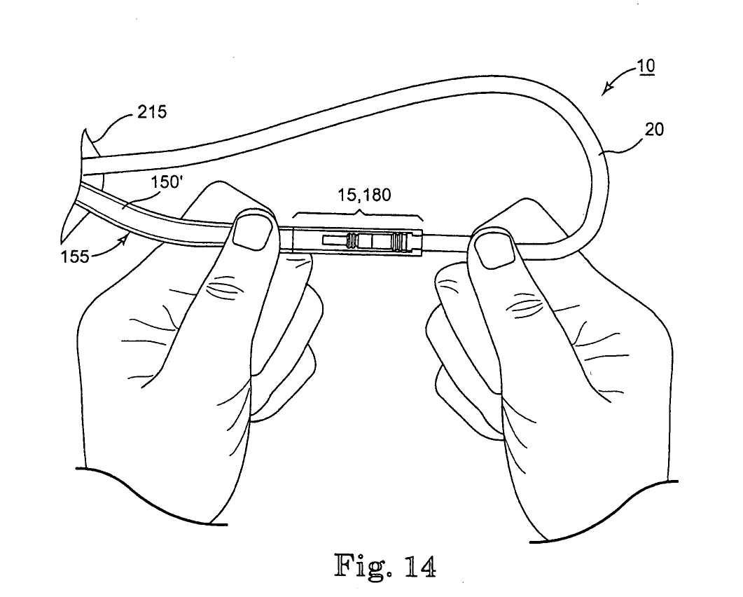

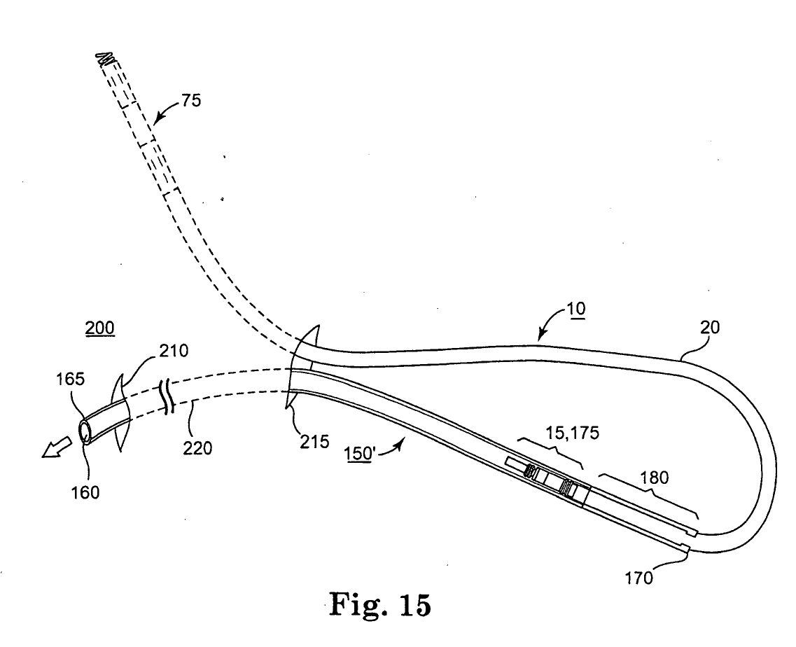

As shown in FIG. 12, the lead distal segment 75 is positioned at a stimulation site and the tissue pathway 220 between the first and second skin incisions 210 and 215 is formed with the sleeve 150′ over the needle shaft 115 in the same manner as described with respect to FIG. 7. The needle shaft is withdrawn from the sheath lumen 160 as shown in FIG. 13, and the lead connector 15 can be inserted laterally (transversely to the plane of the figure) through the sleeve lumen window 180 into the sheath lumen 160 as shown in FIG. 14. Then, the lead connector is preferably (although not necessarily) axially advanced toward the sleeve first end 165 and into the connector receiving chamber 175 of the sleeve lumen 160 to frictionally engage the lead connector 15 with the sleeve body 155 as shown in FIG. 15. The sleeve 150′ may then be retracted through the tissue pathway 220 and drawn out of the first skin incision 210 as shown in FIG. 15 to draw the proximal portion of the medical electrical lead 10 into the second skin incision 215 and through the tissue pathway 220. The lead connector 15 is drawn out of the first skin incision 210 and released laterally through the sleeve lumen window 180 from the sleeve lumen 160. The exposed lead connector 15 can then be connected with an IPG connector or an extension lead connector as described above.

In a further preferred embodiment illustrated in FIGS. 16 and 17, the sleeve body 155 in the connector receiving chamber 175 and/or the sleeve lumen opening 180 of modified sleeve 150′ may be shaped to have connector engagement features defining a sleeve lumen diameter SLD that is narrowed at one or more point to facilitate frictional engagement with the lead connector 15. As described above, alternatively, or additionally, the lumen wall of the sleeve lumen 160 may be coated with a coating 230 of a tacky biocompatible adhesive that provides adhesion with the lead connector 15.

In FIGS. 16 and 17, annular ribs or rings 190 and 195 are formed extending from the sleeve body 155 into the sleeve lumen 160 within the connector receiving chamber 175 and/or the sleeve lumen opening 180 of modified sleeve 150′. The ribs 190 and 195 are shaped to define a SLD that is smaller than the lead connector diameter LCD and is greater than the needle shaft diameter NSD. The distance between the annular ribs is selected in relation to the distance between the lead connector sealing rings 90 and 95.

In FIGS. 18 and 19, the sleeve body 155 is indented inward to form convex resilient bands 190′ and 195′ extending into the sleeve lumen 160 within the connector receiving chamber 175 and/or the sleeve lumen opening 180 of modified sleeve 150′. The bands 190′ and 195′ are shaped to define a SLD that is smaller than the lead connector diameter LCD and is greater than the needle shaft diameter NSD. The distance between the annular bands 190′ and 195′ is also selected in relation to the distance between the lead connector sealing rings 90 and 95.

Thus, exemplary methods of passing a medical electrical lead having a medical electrical lead body extending from a proximal lead connector to a distal stimulation/sense electrode through a tissue pathway to dispose the stimulation/sense electrode in or adjacent a selected stimulation site are described. In one preferred use of the tunneling instruments and methods described above, the stimulation site is urethral tissue structure of the urethra of a patient, the method comprises: (1) making a urethral incision in body tissue accessing the urethral tissue structure; (2) making a skin incision through the skin in an abdominal region of the patient's body; (3) passing the assembled sleeve and needle from one incision to the other incision to dispose the first end extending from the skin incision and the second end extending from the urethral incision; (4) withdrawing the needle from the tissue pathway; (5) coupling the lead connector to the sleeve second end; (6) manipulating the sleeve to draw the lead body through the tissue pathway and the lead connector out of the skin incision; and (7) positioning the stimulation/sense electrode in relation to the tissue structure of the urethra before or after any of steps (2)-(6).

The implantation of a tissue stimulator comprising a medical electrical lead implanted in accordance with the above steps may further comprise: forming a subcutaneous tissue pocket proximate the abdominal skin incision; coupling a tissue stimulation IPG to the lead connector; inserting the IPG in the pocket; and closing the pocket.

In certain embodiments, distal segments of medical electrical lead bodies are shaped to advantageously apply unipolar, bipolar or multi-polar neurostimulation to tissue structure of or adjacent the urethra to effect urethral constriction.

A still further aspect of the invention involves medical electrical leads for placement in the pelvic floor having fixation mechanisms for stabilization of the stimulation/sense electrodes to inhibit dislodgement from a selected stimulation site. In certain embodiments, the fixation mechanisms encourage fibrosis about the lead to chronically stabilize the position of the medical electrical lead and/or stimulation/sense electrode(s). In certain embodiments, the fixation mechanisms are isolated from body tissue during routing of the medical electrical lead through a tissue pathway and then exposed to body tissue to encourage fibrosis.

Similar kits, systems and methods may be practiced for use in the implantation of neural stimulator IMDs to apply neural stimulation to a nerve stimulation electrode of a neural lead positioned in proximity to a nerve in the pelvic region to effect neural stimulation.

The tunneling instruments and methods of the present invention may also advantageously employed in forming other tissue pathways between skin incisions to draw other elongated medical devices or implants therethrough for permanent implantation or temporary use.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments. It will also be understood that there may be other structures, functions and operations ancillary to the typical surgical procedures that are not disclosed and are not necessary to the practice of the present invention.

In addition, it will be understood that specifically described structures, functions and operations set forth in the above-referenced patents can be practiced in conjunction with the present invention, but they are not essential to its practice. It is therefore to be understood, that within the scope of the appended claims, the invention may be practiced otherwise than as specifically described without actually departing from the spirit and scope of the present invention.

1. A tunneling instrument for passing a medical electrical lead subcutaneously between a first and a second skin incision, the lead having a lead body extending between a distal electrode and a proximal lead connector having a lead connector diameter, the tunneling instrument comprising:
an elongated sleeve comprising a sleeve body having a sleeve body length sufficient to extend from the first and second skin incisions and extending from a sleeve first end and a sleeve second end, the sleeve body enclosing a sleeve lumen having a sleeve lumen diameter; and
an elongated needle comprising a needle shaft extending between a needle first end and a needle second end having a needle shaft diameter and a shaft length sufficient to extend from the first and second skin incisions, wherein:
the sleeve lumen diameter is sized to the needle shaft diameter to enable insertion of the needle shaft through the sleeve lumen with the needle second end in proximity to the sleeve second end to form a tunneling instrument adapted to be manipulated to create a subcutaneous tissue pathway between the first and second skin incisions, the sleeve lumen diameter enabling withdrawal of the needle shaft from the sleeve lumen leaving the sleeve extending through the tissue pathway; and
the sleeve lumen diameter is sized to the lead connector diameter at least proximate the sleeve second end to provide frictional engagement of the lead connector against the sleeve body upon insertion of the lead connector into the sleeve lumen so that the sleeve and lead body may be pulled through the tissue pathway to dispose the lead connector outside the first skin incision.

2. The tunneling instrument of claim 1, wherein a sleeve lumen window to the sleeve lumen is formed across the sleeve second end to enable advancement of the lead connector through the sleeve lumen window to frictionally engage the sleeve body.

3. The tunneling instrument of claim 1, wherein a sleeve lumen window to the sleeve lumen is formed as a slot through the sleeve body extending from substantially the sleeve second end toward the sleeve first end, to enable lateral insertion of the lead connector through the slot and axial advancement of the lead connector toward the sleeve first end to frictionally engage the sleeve body.

4. The tunneling instrument of claim 1, further comprising a handle attached to the needle shaft at the needle first end adapted to be grasped to advance the tunneling instrument through tissue to form the tissue pathway, and wherein the sleeve first end abuts the handle when the needle shaft is inserted through the sleeve lumen.

5. The tunneling instrument of claim 1, wherein the lead connector is formed having a resilient sealing ring having a sealing ring diameter exceeding the sleeve lumen diameter, whereby frictional engagement of the lead connector with the sleeve body is attained by compression of the resilient sealing ring within the sleeve lumen.

6. The tunneling instrument of claim 5, wherein a sleeve lumen window to the sleeve lumen is formed across the sleeve second end to enable advancement of the lead connector through the sleeve lumen window into a connector receiving chamber of the sleeve lumen thereby compressing the resilient sealing ring within the sleeve lumen to frictionally engage the sleeve body.

7. The tunneling instrument of claim 5, wherein a sleeve lumen window to the sleeve lumen is formed as a slot through the sleeve body extending from substantially the sleeve second end toward the sleeve first end, to enable insertion of the lead connector through the slot and advancement of the lead connector toward the sleeve first end into a connector receiving chamber of the sleeve lumen thereby compressing the resilient sealing ring within the sleeve lumen to frictionally engage the sleeve body.

8. The tunneling instrument of claim 1, wherein at least a sleeve lumen segment proximate the sleeve distal end is formed having at least one rib extending laterally from the sleeve wall into the sleeve lumen to define the sleeve lumen diameter less than the lead connector diameter.

9. The tunneling instrument of claim 1, wherein at least a sleeve lumen segment proximate the sleeve distal end is formed having at least one resilient band extending laterally from the sleeve wall into the sleeve lumen to define the sleeve lumen diameter less than the lead connector diameter.

10. The tunneling instrument of claim 1, wherein at least a sleeve lumen segment proximate the sleeve distal end is coated with or formed by a material having a tacky surface enhancing engagement with the lead connector.

11. A method of passing an elongated medical device having first and second ends between a first and a second skin incision, the device having a device body diameter proximate the device first end, the method comprising:
providing tunneling instrumentation comprising:
an elongated needle comprising a needle shaft extending from a needle first end and a needle second end and having a needle diameter; and
an elongated sleeve comprising a sleeve body extending from a sleeve first end and a sleeve second end, the sleeve body enclosing a sleeve lumen having a sleeve lumen diameter greater than the needle diameter and smaller than the device body diameter proximate the sleeve second end,
extending the elongated needle through the sleeve lumen to dispose the sleeve body over the needle body and the needle second end in proximity to the sleeve second end to form a tunneling instrument;
advancing the tunneling instrument subcutaneously from one of the first and second skin incision to the other of the first and second skin incision to dispose the sleeve second end proximate the second skin incision and to form a subcutaneous tissue pathway between the first and second skin incision;
withdrawing the elongated needle at least from a sleeve lumen segment proximate the second skin incision;
inserting the device first end into a sleeve lumen segment from the sleeve second end to engage the device first end with the sleeve body;
withdrawing the elongated needle from the sleeve lumen;
pulling the sleeve and device body through the tissue pathway to dispose the device first end outside the first skin incision; and
detaching the device first end from the sleeve lumen.

12. The method of claim 11, wherein a sleeve lumen window to the sleeve lumen is formed across the sleeve second end, and the inserting step comprises advancing the device first end through the sleeve lumen window and into the sleeve lumen to frictionally engage the device first end with the sleeve body.

13. The method of claim 11, wherein a sleeve lumen window to the sleeve lumen is formed as a slot through the sleeve body extending from substantially the sleeve second end toward the sleeve first end, and the inserting step comprises:
inserting the device first end through the slot; and
advancing the device first end toward the sleeve first end into the sleeve lumen to frictionally engage the device first end with the sleeve body.

14. The method of claim 11, wherein the elongated medical device is a medical electrical lead having a lead body extending between a distal electrode and a proximal lead connector having a lead connector diameter

15. The method of claim 14, wherein a sleeve lumen window to the sleeve lumen is formed across the sleeve second end, and the inserting step comprises advancing the lead connector through the sleeve lumen window and into the sleeve lumen to frictionally engage the lead connector with the sleeve body.

16. The method of claim 14, wherein a sleeve lumen window to the sleeve lumen is formed as a slot through the sleeve body extending from substantially the sleeve second end toward the sleeve first end, and the inserting step comprises:
inserting the lead connector through the slot; and
advancing the lead connector toward the sleeve first end into the sleeve lumen to frictionally engage the lead connector with the sleeve body.

17. The method of claim 14, wherein at least a sleeve lumen segment proximate the sleeve distal end is formed having at least one rib extending laterally from the sleeve wall into the sleeve lumen to define the sleeve lumen diameter less than the lead connector diameter.

18. The method of claim 14, wherein at least a sleeve lumen segment proximate the sleeve distal end is formed having at least one resilient band extending laterally from the sleeve wall into the sleeve lumen to define the sleeve lumen diameter less than the lead connector diameter.

19. The method of claim 14, wherein at least a sleeve lumen segment proximate the sleeve distal end is coated with or formed by a material having a tacky surface enhancing engagement with the lead connector.

20. A method of passing a medical electrical lead having a medical electrical lead body extending from a proximal lead connector to a distal stimulation/sense electrode through a tissue pathway to dispose the stimulation/sense electrode in or adjacent urethral tissue structure of the urethra of a patient comprising:
making a urethral incision in body tissue accessing the urethral tissue structure;
making a skin incision through the skin in an abdominal region of the patient's body;
providing an elongated sleeve comprising a sleeve body extending from a sleeve first end and a sleeve second end, the sleeve body enclosing a sleeve lumen having a sleeve lumen diameter smaller than the sealing ring diameter at least proximate the sleeve second end to enable frictional engagement of the sleeve body with the resilient sealing ring;
providing an elongated needle comprising a needle shaft extending from a needle first end and a needle second end and having a needle diameter smaller than the sleeve lumen diameter,
advancing the needle shaft through the sleeve lumen to dispose the sleeve body over the needle body and the needle second end in proximity to the sleeve second end to form a tunneling instrument;
advancing the tunneling instrument through tissue to form a subcutaneous tissue pathway between the urethral and abdominal skin incisions;
withdrawing the needle shaft from at least a sleeve lumen segment proximate the urethral skin incision;
inserting the lead connector into a sleeve lumen segment of the sleeve body extending from the urethral skin incision; and
pulling the sleeve and lead body through the tissue pathway to dispose the lead connector outside the abdominal skin incision.

21. The method of claim 20, further comprising:
positioning the stimulation/sense electrode in relation to the tissue structure of the urethra.
